(19) United States
(12) Patent Application Publication
(10) Pub. No.: US 2020/0353007 A1
(43) Pub. Date: Nov. 12, 2020

(54) Pharmacological Composition for Prevention or Treatment of Lupus, Comprising Mesenchymal Stem Cell-Derived Secretome

(71) Applicant: K-BIO CELF INC., Gyeonggi-do (KR)

(72) Inventors: Yong Beom Park, Seoul (KR); Chin Hee Mun, Gyeonggi-do (KR)

(21) Appl. No.: 16/641,986

(22) PCT Filed: Aug. 27, 2018
(86) PCT No.: PCT/KR2018/009873

§ 371 Date: Feb. 25, 2020

(30) Foreign Application Priority Data
Aug. 25, 2017 (KR) 10-2017-0108171

(51) Int. Cl. A61K 35/28; A61K 38/17

(52) U.S. Cl. CPC A61K 35/28; A61K 38/1709

(57) ABSTRACT

The present disclosure relates to a composition for various applications, which is capable of effectively preventing, ameliorating or treating lupus using a secretome derived from mesenchymal stem cells.

The secretome derived from mesenchymal stem cells according to the present disclosure may significantly decrease mortality and the amount of proteinuria, and may increase body weight, decrease the expression of serum creatinine, and inhibit glomerular, coronary and vascular damage in kidney tissue. Furthermore, the secretome may reduce the size of an enlarged spleen and reduce the number of splenocytes and CD4-positive T cells. In addition, the secretome may increase the expression of the anti-inflammatory cytokines IL-10 and TGF-β1 in serum, and decrease the expression of anti-dsDNA antibody. In the mechanism thereof, the secretome may effectively prevent, ameliorate or treat lupus nephritis and, furthermore, lupus, by increasing the activity of Treg cells and inhibiting the activity of the inflammatory cells Th1 and Th2 cells, B cells, dendritic cells and inflammatory macrophages.

### TECHNICAL FIELD

The present disclosure relates to a composition for various applications, which is capable of effectively preventing, ameliorating or treating lupus using a secretome derived from mesenchymal stem cells.

### BACKGROUND ART

Systemic lupus erythematosus (SLE), also called ‘lupus’, is a chronic autoimmune inflammatory disease with complex clinical features. It is an autoimmune disease which is caused by inflammation due to the abnormal immune response and autoantibody production of hyper-activated B cells and T cells, which results in immune complex deposition, and affects various organs of the whole body.

Lupus nephritis is characterized by organ involvement frequently occurring in systemic lupus erythematosus patients, and is an inflammation of the kidney caused by the deposition of inflammatory cells and immune complexes. It is a serious organ involvement that, if not properly treated, directly contributes to the prognosis and mortality of patients with systemic lupus erythematosus that leads to chronic kidney failure due to kidney function impairment (Agrawal et al., 2006). Although many immune and non-immune factors contribute to the manifestation of lupus nephritis, the production of autoantibodies against nuclear antigens and endogenous antigens and the formation of glomerular immune deposits play an important role in the development of lupus nephritis (Deocharan et al. 2002; Lefkowith and Gilkeson, 1996). Several studies reported that anti-DNA antibodies are directly involved in the development of lupus nephritis by binding directly to cross-reactive antigens or indirectly to glomerular basement membrane components (Yung and Chan, 2008). Furthermore, cytokines and chemoattractants induced by kidney cells and invasive immune cells exacerbate immune complex-mediated kidney injury (Aringer and Smolen, 2005; Kulkarni and Anders, 2008). Lupus treatment is currently focused on immunosuppressants such as corticosteroids, cyclophosphamide, azathioprine, and mycophenolate mofetil (Waldman and Appel, 2006). However, these drugs involve dangerous side effects that make patients susceptible to infection and cancer, together with the biotoxicity of the drugs themselves (Radis et al. 1995). For this reason, there is increasing interest in the development of low-toxicity drugs for controlling immune complex formation and deposition, as well as drugs that directly counter inflammatory responses.

Mesenchymal stem cells are undifferentiated adult stem cells which are found in the differentiated cells of tissues or organs, can be isolated from various tissues in the human body, such as bone marrow, fat and muscle, and have the self-renewal ability to renew themselves. In addition, the mesenchymal stem cells can easily proliferate in vitro and can differentiate into various tissue cells, such as adipocytes, bone cells, chondrocytes and myocytes, and thus previous stem cell studies have been focused on reproduction studies based on the differentiation potential of the stem cells.

The immunomodulatory function of mesenchymal stem cells, which has recently been revealed by several studies, protects hematopoietic stem cells from damage caused by immune responses, and acts at each stage of the immune response to exhibit an immunomodulatory effect, resulting in immune response suppression and anti-inflammatory response. This immunomodulatory function occurs through interaction with various immune cells, such as natural killer (NK) cells, dendritic cells, macrophages, T cells and B cells. The anti-inflammatory effect of mesenchymal stem cells is explained by a paracrine mechanism by which damaged tissue is repaired by various growth factors and proteins secreted from stem cells, but studies related to the treatment of inflammatory diseases using the paracrine effect are still insufficient.

Meanwhile, secretomes can be produced and synthesized in large amounts from allogeneic cell lines whose characterization, contamination analysis and quality control for clinical use have all been completed. Thus, the secretomes are biological agents that can easily overcome the lack of donor cells or medical problems, unlike cell therapy agents for cell replacement.

### DISCLOSURE

### Technical Problem

One object of the present disclosure is to provide a composition capable of effectively preventing, ameliorating or treating lupus using a secretome derived from a culture of mesenchymal stem cells, which pose no ethical problems and have no immunogenicity.

However, the technical objects to be achieved by the present disclosure are not limited to the above-mentioned object, and other objects that are not mentioned herein will be clearly understood by those skilled in the art from the following description.

### Technical Solution

One embodiment of the present disclosure is directed to a pharmaceutical composition for preventing or treating lupus, containing a secretome derived from mesenchymal stem cells as an active ingredient.

In the present disclosure, the “mesenchymal stem cells” refer to multipotent undifferentiated cells derived from adult cells of mammals including humans, preferably humans, and may be derived from various adult cells of, for example, bone marrow, blood, brain, skin, fat (i.e., adipose tissue or adipocytes), umbilical cord blood or umbilical cord Wharton's jelly.

In addition, in the present disclosure, the “secretome” means the sum of protein components among the components secreted from the mesenchymal stem cells. The secretome refers to components released by cells into the extracellular environment after the transcription, translation and post-translational modification of genes in the cells. Typical expression markers of the secretome correspond to growth factors, such as EGF and VEGF, and extracellular matrix proteins such as collagen and fibronectin.

In the present disclosure, the secretome may be isolated from a culture obtained by culturing the mesenchymal stem cells.

In the present invention, a method for culturing the mesenchymal stem cells may be performed by culturing the mesenchymal stem cells in mesenchymal stem cell culture medium for 24 to 96 hours, and then culturing the cells in serum-free medium for 24 to 72 hours.

Here, the composition of the mesenchymal stem cell culture medium is not particularly limited, but the mesenchymal stem cell culture medium may be serum medium. For example, the mesenchymal stem cell culture medium may be a Dulbecco's modified Eagle's medium (DMEM) or RPMI-1640 medium containing 5 to 15 wt % of fetal bovine serum (FBS) and 0.05 to 0.2 mM of mercaptoethanol, or may be serum-free medium such as StemPro medium, MSCGro medium, MesenCult medium or NutriStem medium, but is not limited thereto, and any medium may be used without limitation as long as it is a medium that may be used for culturing of mesenchymal stem cells in the art.

In addition, the serum-free medium may be phenol red- and antibiotic-free Dulbecco's modified Eagle's medium (DMEM), but is not limited thereto, and any medium may be used without limitation as long as it is fetal bovine serum-free medium that may be used for culturing of mesenchymal stem cells, including a medium that may be used for clinical-grade cell culture in the art.

In the present invention, the secretion amount and components of the secretome may differ depending on the culturing conditions of the mesenchymal stem cells, and conventional culturing is performed under normal oxygen partial pressure (about 20% by volume of oxygen). However, the in vivo environment is under low-oxygen partial pressure, and when this environment is provided in vitro and stem cells are cultured therein, the growth, differentiation and angiogenic potential of the cells may be improved, and thus the therapeutic effect of the stem cells may also be enhanced. Therefore, in the present disclosure, the secretome may be obtained not only by culturing the mesenchymal stem cells under normal oxygen culture conditions (20% by volume of 02), but also by culturing the mesenchymal stem cells under low-oxygen culture conditions (0.5 to 1% by volume of 02).

In addition, in the present invention, as the secretome, it is preferable to use a polymeric concentrate obtained after centrifuging a culture, obtained by culturing as described above, at 500 to 1,500×g, and recovering the supernatant, because this polymeric concentrate can inhibit the expression of inflammatory cytokines and activate the expression of anti-inflammatory cytokines, thus further suppressing immune response.

In one embodiment of the present disclosure, the polymeric concentrate may be obtained through a step of filtering the supernatant, obtained by centrifugation, through a 0.1 to 0.3 μm filter, preferably a 0.2 μm filter, and a step of filtering molecules of 3 kDa or less in size.

Here, the method of filtering the molecules of 3 kDa or less in size may be performed by diafiltration using a tangential flow filtration (TFF) system. In addition, in the present disclosure, during the diafiltration, the supernatant may be concentrated at 0 and 5° C. while it is replaced and diluted with water for injection by means of a peristaltic tubing pump.

In other embodiments of the present disclosure, the polymeric concentrate may be obtained by concentrating the active ingredient of the supernatant, obtained by centrifugation, while reacting the supernatant with a polar alcohol solvent.

As the polar alcohol solvent, there may be used one or two or more selected from among a lower alcohol having 1 to 6 carbon atoms, a dilution of the alcohol, for example, a 95% or 90% aqueous solution of the alcohol, and acetone which is reduced to isopropyl alcohol by a reducing agent.

In the present disclosure, the aqueous solution of the alcohol refers to a dilution of the alcohol, and may include, for example, 95% ethanol, 90% ethanol, or the like.

In the present disclosure, the polar alcohol solvent is preferably mixed with the supernatant in an amount of 2 to 5 times the weight of the supernatant, because only an active ingredient of the polymeric concentrate in the supernatant may be effectively concentrated.

In addition, in the present disclosure, the reaction between the supernatant and the polar alcohol solvent is preferably performed at −30 to 0° C. for 5 to 500 minutes.

According to one example of the present invention, 100% alcohol may be added to the supernatant, obtained by centrifugation of the culture obtained by culturing the mesenchymal stem cells as described above, and the resulting mixture may be left to stand at −30 to 0° C. for 5 to 500 minutes. Thereafter, the resulting mixture may be centrifuged, and then 90% alcohol may be added to the precipitate, followed by further centrifugation. Alternatively, the precipitate obtained after centrifugation may be added to and suspended in sterile water, and then lyophilized.

The present disclosure may, if necessary, further include a step of freeze drying the polymeric concentrate, obtained as described above, for 6 to 10 hours. In the present disclosure, the polymeric concentrate of the secretome may be obtained as an agent in powder form by the freeze-drying.

In the present disclosure, the secretome derived from mesenchymal stem cells, obtained as described above, can effectively prevent, ameliorate or treat lupus, especially lupus nephritis.

In the present invention, the “lupus” refers to an autoimmune disease or disorder in which antibodies that affect connective tissues are involved. The main forms of lupus are systemic diseases, including all types of lupus that can affect multiple internal organs (kidneys, lungs, heart, central and peripheral nerves, gastrointestinal tract, bone marrow, liver, spleen, peripheral blood cells, skin, mucous membranes, scalp, and the like).

Lupus nephritis in the present invention is glomerulonephritis that frequently occurs in systemic lupus erythematosus, involves antigen-antibody complex deposition in the vasculature and basement membranes, hematuria and uremia, and can also show a fulminant course leading to death within a few weeks, but shows a chronic progressive course in most cases. However, if lupus nephritis is not treated properly, kidney damage can progress and lead to chronic kidney failure, and hence lupus nephritis has a very important effect on the prognosis of lupus patients. Lupus is an autoimmune disease occurring in patients with genetic predisposition. It is a disease that occurs because immune cells, on which environmental factors such as ultraviolet rays or bacterial or viral infections act, excessively react and autoantibodies produced by the immune cells recognize our bodies as enemies, and attack and damage the multiple organs of our bodies. Lupus nephritis refers to a disease in which various autoantibodies or immune complexes that are excessively present in blood are deposited in kidney glomeruli and inflammatory cells penetrate into the glomeruli and cause inflammation and kidney glomerular damage. In lupus nephritis, kidney tissue is destroyed and abnormal findings such as proteinuria and hematuria appear in urine tests. When a large amount of protein in blood comes out while proteinuria is aggravated, the blood components are released into tissues and cause the accumulation of body fluid, which causes weight gain and swelling, resulting in swelling of legs, ankles and hands, which is the first symptom of lupus nephritis. In the majority of patients with lupus nephritis, atherosclerosis is more likely to occur, and hence symptoms such as hypertension, hyperlipidemia and hyperglycemia occur.

The “preventing” or “prevention” in the present disclosure refers to a decrease in the occurrence of pathological cells or the extent of cell damage or loss in an animal. The preventing may be complete or partial. In this case, the preventing may refer to a phenomenon in which the occurrence of pathological cells or abnormal immune response in a subject decreases compared when the to composition for preventing and treating lupus is not used.

In the present invention, the “treating” or “treatment” refers to any clinical intervention in an attempt to alter the natural course of the subject or cell to be treated, and can be performed either for prophylaxis or during the course of clinical pathology. Desirable effects of treatment may include preventing occurrence or recurrence of disease, or alleviating symptoms, or diminishing any direct or indirect pathological consequences of the disease, or decreasing the rate of disease progression, or ameliorating or palliating the disease state, or improving prognosis. That is, the treatment may be interpreted as encompassing all actions that improve or completely cure the symptoms of lupus by the composition.

In the present disclosure, the pharmaceutical composition may be in the form of capsule, tablet, granule, injection, ointment, powder or beverage, and the pharmaceutical composition may be for administration to humans.

For use, the pharmaceutical composition of the present disclosure may be formulated in the form of, but not limited to, oral preparations, such as powders, granules, capsules, tablets, and aqueous suspensions, as well as external preparations, suppositories, and sterile injectable solutions, according to the respective conventional methods. The pharmaceutical composition of the present disclosure may contain pharmaceutically acceptable carriers. Pharmaceutically acceptable carriers that may be used for oral administration include binders, lubricants, disintegrants, excipients, solubilizers, dispersants, stabilizers, suspending agents, pigments, flavorings, and the like, and pharmaceutically acceptable carriers that may be used for injection include buffers, preservatives, analgesics, solubilizers, isotonic agents, stabilizers, and the like. Pharmaceutically acceptable carriers that may be used for topical administration include bases, excipients, lubricants, preservatives, and the like. The formulation of the pharmaceutical composition of the present disclosure may be prepared in various forms by mixing with the pharmaceutically acceptable carriers as described above. For example, for oral administration, the pharmaceutical composition may be prepared in the form of tablets, troches, capsules, elixir, suspensions, syrups, wafers, and the like, and for injection, the pharmaceutical composition may be presented in unit dose ampoules or multi-dose containers. In addition, the pharmaceutical composition may be formulated as solutions, suspensions, tablets, capsules, sustained-release preparations, or the like.

Meanwhile, examples of carriers, excipients and diluents suitable for formulation include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil. In addition, the pharmaceutical composition of the present disclosure may further contain a filler, an anticoagulant, a lubricant, a wetting agent, a flavoring, an emulsifier, a preservative, or the like.

The routes of administration of the pharmaceutical composition according to the present disclosure include, but are not limited to, oral, intravenous, intramuscular, intra-arterial, intramedullary, intradural, intracardiac, transdermal, subcutaneous, intraperitoneal, intranasal, gastrointestinal, topical, sublingual and intrarectal routes. Oral or parenteral administration is preferred.

In the present disclosure, “parenteral” includes subcutaneous, transdermal, intravenous, intramuscular, intra-articular, intra-synovial, intrasternal, intradural, intra-lesional and intra-cranial injection or infusion techniques. The pharmaceutical composition of the present disclosure may also be formulated as suppositories for intrarectal administration.

The pharmaceutical composition of the present disclosure may vary depending on various factors, including the activity of specific compounds used, the patient's age, body weight, general health, sex, diet, the period of administration, the route of administration, excretion rate, the drug content, and the severity of a specific disease to be prevented or treated. The dose of the pharmaceutical composition may be suitably selected by a person skilled in the art depending on the patient's condition, body weight, the severity of the disease, the form of drug, and the route and period of administration, and may be 0.0001 to 50 mg/kg/day or 0.001 to 50 mg/kg/day. The pharmaceutical composition may be administered once or several times a day. The dose is not intended to limit the scope of the present disclosure in any way. The pharmaceutical composition according to the present disclosure may be formulated as pills, sugar-coated tablets, capsules, liquids, gels, syrups, slurries, or suspensions.

Another embodiment of the present disclosure is directed to a food composition for preventing or ameliorating lupus, containing a secretome derived from mesenchymal stem cells.

Detailed description of the mesenchymal stem cells and the secretome in the present disclosure is the same as described above for the pharmaceutical composition, and will be omitted below in order to avoid excessive complexity of the description.

The food composition of the present disclosure may be prepared as various foods, for example, beverages, gums, teas, vitamin complexes, powders, granules, tablets, capsules, confectionery, cakes, bread, and the like. The food composition of the present disclosure contains the secretome derived from mesenchymal stem cells having little or no toxicity and side effects, and thus may be used with confidence even when it is administered for a long period of time for preventive purposes.

When the secretome of the present disclosure or a polymeric concentrate including the same is contained in the food composition, it may be added in an amount of 0.1 to 50 wt % based on the total weight of the food composition.

When the food composition is prepared as a beverage, there is no particular limitation, except that the beverage contains the food composition at the indicated percentage. The beverage may additionally contain various flavorings or natural carbohydrates, like conventional beverages. Specifically, the natural carbohydrates include monosaccharides such as glucose, disaccharides such as fructose, polysaccharides such as sucrose, conventional sugars such as dextrin, cyclodextrin or the like, and sugar alcohols such as xylitol, sorbitol, erythritol or the like. Examples of the flavorings include natural flavorings (thaumatin, stevia extracts, such as rebaudioside A, glycyrrhizin, etc.) and synthetic flavorings (saccharin, aspartame, etc.).

In addition, the food composition of the present disclosure may contain various nutrients, vitamins, minerals (electrolytes), flavorings such as synthetic flavorings and natural flavorings, colorants, pectic acid and its salt, alginic acid and its salt, organic acids, protective colloidal thickeners, pH adjusting agents, stabilizers, preservatives, glycerin, alcohol, carbonizing agents that are used in carbonated beverages, etc.

Such components may be used individually or in combination. Although the content of such additives is not of great importance, it is generally selected in a range of 0.1 to about 50 parts by weight based on 100 parts by weight of the food composition of the present disclosure.

Still another embodiment of the present disclosure is directed to a method for preventing or treating lupus, including a step of administering a target subject the secretome derived from mesenchymal stem cells provided in the present disclosure or the pharmaceutical composition provided in the present disclosure in order to prevent or treat lupus.

The “target subject” in the present disclosure refers to a subject who has or is at high risk of lupus.

The dosage, schedule, and route of administration of the secretome provided in the present disclosure may be determined according to the size and condition of the subject and to standard ro pharmaceutical practice. Exemplary routes of administration include intravenous, intra-arterial, intraperitoneal, intrapulmonary, intravascular, intramuscular, intratracheal, subcutaneous, intraocular, intrathecal and transdermal routes.

The dose of the secretome administered to the subject may vary depending, for example, on the particular type of secretome administered, the route of administration and the particular type and disease stage of lupus being treated. The amount should be sufficient to produce a desired response, such as a therapeutic response against lupus, without severe toxicity or adverse events. The magnitude of this effect can be measured using standard methods, such as in vitro assays with purified enzyme, cell-based assays, animal models, or human testing.

In addition, in the present disclosure, the secretome may be formulated and administered as oral preparations, such as powders, granules, capsules, tablets and aqueous suspensions, as well as external preparations, suppositories, and sterile injectable solutions, according to the respective conventional methods.

In addition, in the present disclosure, the secretome may be administered together with pharmaceutically acceptable carriers. Pharmaceutically acceptable carriers that may be used for oral administration include binders, lubricants, disintegrants, excipients, solubilizers, dispersants, stabilizers, suspending agents, pigments, flavorings, and the like, and pharmaceutically acceptable carriers that may be used for injection include buffers, preservatives, analgesics, solubilizers, isotonic agents, stabilizers, and the like. Pharmaceutically acceptable carriers that may be used for topical administration include bases, excipients, lubricants, preservatives, and the like. In addition, in the present disclosure, the secretome may be prepared in various forms by mixing with the pharmaceutically acceptable carriers. For example, for oral administration, the secretome may be prepared in the form of tablets, troches, capsules, elixir, suspensions, syrups, wafers, and for injection, the secretome may be presented in in unit dose ampoules or multi-dose containers. In addition, the secretome may be formulated as solutions, suspensions, tablets, capsules, sustained-release preparations, or the like.

Meanwhile, examples of carriers, excipients and diluents suitable for formulation include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil. In addition, a filler, an anticoagulant, a lubricant, a wetting agent, a flavoring, an emulsifier, a preservative, or the like may additionally be included.

### Advantageous Effects

The secretome derived from mesenchymal stem cells according to the present disclosure can significantly reduce the amount of proteinuria, and can increase body weight, decrease the expression of serum creatinine, and inhibit glomerular, coronary and vascular damage in kidney tissue. Furthermore, the secretome can reduce the size of an enlarged spleen and reduce the number of splenocytes and CD4-positive T cells. In addition, the secretome can increase the expression of the anti-inflammatory cytokines IL-10 and TGF-β1 in serum, and decrease the expression of anti-dsDNA antibodies. The secretome derived from mesenchymal stem cells according to the present disclosure can effectively prevent, ameliorate or treat lupus nephritis and, furthermore, lupus, by increasing the activity of regulatory T cells (Treg) and inhibiting the activity of the inflammatory cells Th1 and Th2 cells, B cells, dendritic cells, and inflammatory macrophages.

It is to be understood that the effects of the present disclosure are not limited to the above-described effects and include all the effects that can be deduced from the configurations of the disclosure described in the detailed description of the disclosure or the appended claims.

### DESCRIPTION OF DRAWINGS

FIG. 1 schematically shows an experimental design in which lupus nephritis mouse models are treated with either a secretome, isolated and concentrated according to one embodiment of the present disclosure, or adipose-derived mesenchymal stem cells (MSCs) as a control, in Example 2.

FIG. 2 graphically shows the results of comparing the survival rate of mice after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 2. The mortality of lupus nephritis mouse models in the group treated with the secretome decreased compared to that in the untreated group.

FIG. 3 graphically shows the results of measuring proteinuria after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 2. The amount of proteinuria in the group treated with the secretome significantly decreased compared to that in the untreated group, and the level of decrease in proteinuria in the group treated with the secretome was similar to that in the group treated with methylprednisolone (*,p<0.05; **,p<0.01; ***,p<0.001).

FIG. 4 graphically shows the results of measuring serum creatinine after treating lupus nephritis mouse models with each of a secretome, isolated and concentrated according to one embodiment of the present disclosure, and methylprednisolone as a positive treatment control, in Example 2. The expression of serum creatinine in the group treated with the secretome significantly decreased compared to that in the untreated group (*,p<0.05; ***,p<0.001).

FIG. 5 shows images of PAS-stained kidney tissue, obtained after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 3.

FIG. 6 graphically shows the results of evaluating the extent of kidney glomerular damage after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 3. Glomerular damage in the group treated with the secretome was significantly inhibited compared to that in the untreated group (***,p<0.001).

FIG. 7 graphically shows the results of evaluating the extent of kidney tubular damage after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 3. Kidney tubular damage in the group treated with the secretome was significantly inhibited compared to that in the untreated group (***p<0.001).

FIG. 8 graphically shows the results of evaluating the extent of kidney vascular damage after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 3. Kidney vascular damage in the group treated with the secretome was significantly inhibited compared to that in the untreated group (*, p<0.05).

FIG. 9 graphically shows the results of measuring the expression levels of IgG and C3 in kidney tissue after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 3. The fluorescence intensities of IgG and C3 in the group treated with the secretome significantly decreased compared to those in the untreated group (***,p<0.001).

FIG. 10 depicts images showing the size of spleen after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 4.

FIG. 11 graphically shows the results of measuring changes in the weight of spleen after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 4.

FIG. 12 graphically shows the results of measuring changes in the number of splenocytes after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 4. The number of splenocytes in the group treated with the secretome significantly decreased compared to that in the untreated group (*,p<0.05; **,p<0.01).

FIG. 13 graphically shows the results of measuring changes in the number of CD4+ T cells in spleen tissue after treating lupus nephritis mouse models with each of a secretome, isolated and concentrated according to one embodiment of the present disclosure, and methylprednisolone as a positive treatment control, in Example 5. The number of CD4+ T cells in the group treated with the secretome significantly decreased compared to that in the untreated group (*,p<0.05; **,p<0.01).

FIG. 14 graphically shows the results of analyzing changes in the expression level of CD4+Foxp3+ cells (regulatory T cells) among mouse splenocytes after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 6. The expression level of CD4+Foxp3+ cells in the group treated with the secretome significantly increased compared to that in the untreated group (*,p<0.05).

FIG. 15 graphically shows the results of analyzing changes in the expression level of CD4+CD25+Foxp3+PD-1+ cells (regulatory T cells) among mouse splenocytes after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 6. The expression level of CD4+CD25+Foxp3+PD-1+ cells in the group treated with the secretome significantly increased compared to that in the untreated group (*,p<0.05; **,p<0.01).

FIG. 16 graphically shows the results of analyzing changes in the expression level of CD4+IFN-γ+ cells (Th1 cells) among mouse splenocytes after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 6.

FIG. 17 graphically shows the results of analyzing changes in the expression level of CD4+IL-4+ cells (Th2 cells) among mouse splenocytes after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 6. The expression level of CD4+IL-4+ cells in the group treated with the secretome significantly decreased compared to that in the untreated group (*,p<0.05).

FIG. 18 graphically shows the results of analyzing changes in the expression level of CD4+IL-17A+ cells (Th17) cells among mouse splenocytes after treating lupus nephritis mouse models with each of a secretome, isolated and concentrated according to one embodiment of the present disclosure, and methylprednisolone as a positive treatment control, in Example 6.

FIG. 19 graphically shows the results of analyzing changes in the expression level of CD19+CD138+ cells (B cells) among mouse splenocytes after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 7. The expression level of CD19+CD138+ B cells in the group treated with the secretome significantly decreased compared to that in the untreated group (***,p<0.001).

FIG. 20 graphically shows the results of analyzing changes in the expression level of CD11c+CD86+ cells (dendritic cells) among mouse splenocytes after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 8. The expression level of CD11c+CD86+ dendritic cells in the group treated with the secretome significantly decreased compared to that in the untreated group (*,p<0.05; ***,p<0.001).

FIG. 21 graphically shows the results of analyzing changes in the expression level of CD11c+MHCII+ cells (dendritic cells) among mouse splenocytes after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 8. The expression level of CD11c+MHCII+ dendritic cells in the group treated with the secretome significantly decreased compared to that in the untreated group (**, p<0.01; ***,p<0.001).

FIG. 22 graphically shows the results of analyzing changes in the expression level of F4/80+CD86+ cells (inflammatory macrophages) among mouse splenocytes after treating lupus nephritis mouse models with each of a secretome, isolated and concentrated according to one embodiment of the present disclosure, and methylprednisolone as a positive treatment control, in Example 9. The expression level ofF4/80+CD86+ macrophages in the group treated with the secretome significantly decreased compared to that in the untreated group (**,p<0.01).

FIG. 23 graphically shows the results of measuring the number of total lymphocytes in kidney tissue after treating lupus nephritis mouse models with each of a secretome, isolated and concentrated according to one embodiment of the present disclosure, and methylprednisolone as a positive treatment control, in Example 10. The number of total lymphocytes in kidney tissue in the group treated with the secretome significantly decreased compared to that in the untreated group (***,p<0.001).

FIG. 24 graphically shows the results of analyzing changes in the expression level of IL-17A in mouse serum after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 11. The expression level of IL-17A in serum in the group treated with the secretome significantly decreased compared to that in the untreated group (*,p<0.05).

FIG. 25 graphically shows the results of analyzing changes in the expression level of IL-6 in mouse serum after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 11. The expression level of IL-6 in serum in the group treated with the secretome significantly increased compared to that in the untreated group (**,p<0.01).

FIG. 26 graphically shows the results of analyzing changes in the expression level of IL-10 in mouse serum after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 11. The expression level of IL-10 in serum in the group treated with the secretome significantly increased compared to that in the untreated group (**,p<0.05).

FIG. 27 graphically shows the results of analyzing changes in the expression level of TGF-β1 in mouse serum after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 11. The expression level of TGF-β1 in serum in the group treated with the secretome significantly increased compared to that in the untreated group (*,p<0.05).

FIG. 28 graphically shows the results of analyzing changes in the expression level of anti-dsDNA in mouse serum after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 12. The expression level of anti-dsDNA in serum in the group treated with the secretome significantly decreased compared to that in the untreated group (*,p<0.05).

FIG. 29 graphically shows the results of analyzing the changes in mouse body weight depending on the treatment period after treating lupus nephritis mouse models with each of a secretome, isolated and concentrated according to one embodiment of the present disclosure, and adipose-derived mesenchymal stem cells (AD-MSCs) or methylprednisolone as a positive treatment control, in Example 13. The mouse body weight in the group treated with the secretome increased as the treatment period increased.

### BEST MODE

One embodiment of the present disclosure is directed to a pharmaceutical composition for preventing or treating lupus, containing a secretome derived from mesenchymal stem cells.

### Mode for Disclosure

Hereinafter, the present disclosure will be described in more detail with reference to Examples. These Examples are merely to illustrate the present disclosure in detail, and it will be obvious to those skilled in the art that the scope of the present disclosure according to the subject matter of the present disclosure is not limited by these Examples.

### EXAMPLES

### [Example 1] Preparation of Secretome Derived from Mesenchymal Stem Cells

1. Reagents and Chemical Products

DMEM (Dulbecco modified Eagle's medium-low glucose), FBS (fetal bovine serum), penicillin/streptomycin and 2-mercaptoethanol (X1000) were purchased from Invitrogen Corp.

2. Obtaining of Mesenchymal Stem Cells from Human Adipocytes

Adipose-derived human mesenchymal stem cells at an early passage were obtained from the Cell Therapy Center of Yonsei University that complies with the Korean Food and Drug Administration guidelines [GMP (Pharmaceutical Manufacturing Quality Control Standards)], and the cells were cultured. Mesenchymal stem cell culture medium (DMEM low glucose supplemented with 10% FBS and 0.1 mM mercaptoethanol) was placed in a culture dish under human mesenchymal stem cell culture conditions clinically approved by the FDA, and the cells were cultured in the medium for 72 to 86 hours. The medium was replaced every 2 to 3 days, and the cells were passaged to a confluence of 70 to 85%. The cells at passage 5 were used in the study.

3. Culturing of Mesenchymal Stem Cells

Mesenchymal stem cells were cultured to a confluence of 80% and repeatedly washed four times or more with PBS buffer to remove protein components such as fetal bovine serum. Next, the cells were cultured with a serum-free medium (DMEM-low glucose) not containing antibiotic and fetal bovine serum for 48 hours, and then the cell culture was recovered.

4. Isolation and Concentration of Secretome from Culture of Mesenchymal Stem Cells

The culture obtained by culturing the mesenchymal stem cells in large scale as described above was centrifuged once at 1000×g to remove the cell residue. Thereafter, large particles such as cell debris were filtered out through a 0.2 μm filter, and molecules of 3 kDa or less in size were filtered by a diafiltration system using tangential flow filtration (TFF) capsules (PALL, Minimate TFF capsules). During the filtration, the culture was concentrated at 4° C. while it was continuously replaced and diluted with water for injection (saline solution or Ringer's solution) by a peristaltic tubing pump. The concentration of protein in the concentrated culture was determined by refractometer measurement and Bradford reagent, and the concentrated culture was stored at −80° C. until the experiment was started.

### [Example 2] Survival Rate of Lupus-Induced Mouse Models, Change in Proteinuria, and Change in Serum Creatinine Concentration

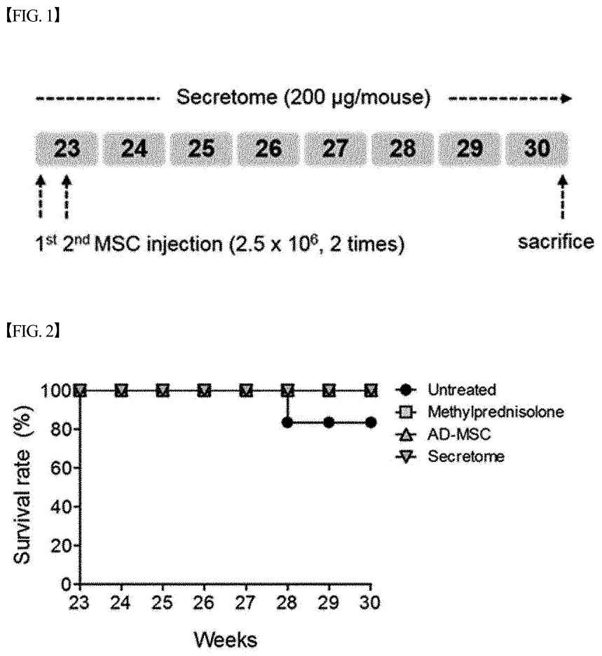

In order to evaluate the lupus nephritis therapeutic effect of the secretome isolated and concentrated in Example 1 above, an experiment was performed as shown in FIG. 1. Specifically, the secretome isolated and concentrated in Example 1 was injected intraperitoneally into lupus nephritis mouse ((NZB/NZW) F1) models (23 weeks of age)) three times a week at a dose of 200 μg/mouse. Then, the survival rate of the mice, proteinuria and the concentration of creatinine in the serum were measured, and the results of the measurement are graphically shown in FIGS. 2 to 4.

Proteinuria was measured twice a week during the experimental period in the spot urine collected from each mouse using an albumin reagent strip (URiSCA; Yeongdong Pharm., Korea). Proteinuria was expressed semi-quantitatively: 0=none or trace; 1+=100 mg/dL or less; 2+=300 mg/dL or less; 3+=2,000 mg/dL or less; and 4+=2,000 mg/dL or more.

In addition, the concentration of creatinine in the serum was measured using a BioAssay Systems QuantiChrom creatinine assay kit by adding a mixed reagent to 30 μl of the serum, and then immediately measuring the OD value, and after 5 minutes, measuring the OD value once more. Thereafter, the concentration of creatinine in the serum was calculated using the following equation:

ODsample 5-OD sample 0/OD STD 5-ODSTD 0×STD (mg/dL).

However, in order to compare the therapeutic effect of the secretome according to the present disclosure, the mice of the negative control group were untreated, and as a positive treatment control, Solumedrol® (methylprednisolone), which has been used as a lupus treatment drug, was injected, or 5×10⁶ human adipose-derived stem cells contained in 100 μl of PBS were injected into the tail veins of the lupus nephritis mouse models (23 weeks old).

As shown in FIG. 2, the mortality of the lupus nephritis mouse models of the group treated with the secretome according to the present disclosure decreased compared to that of the untreated group.

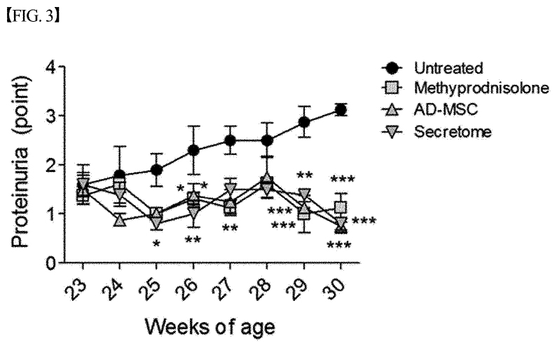

In addition, as shown in FIG. 3, proteinuria increased in the untreated group, but significantly decreased in the group treated with the secretome according to the present disclosure, and the extent of the decrease was similar to that in the group treated with the mesenchymal stem cells or the group treated with the standard lupus treatment drug methylprednisolone.

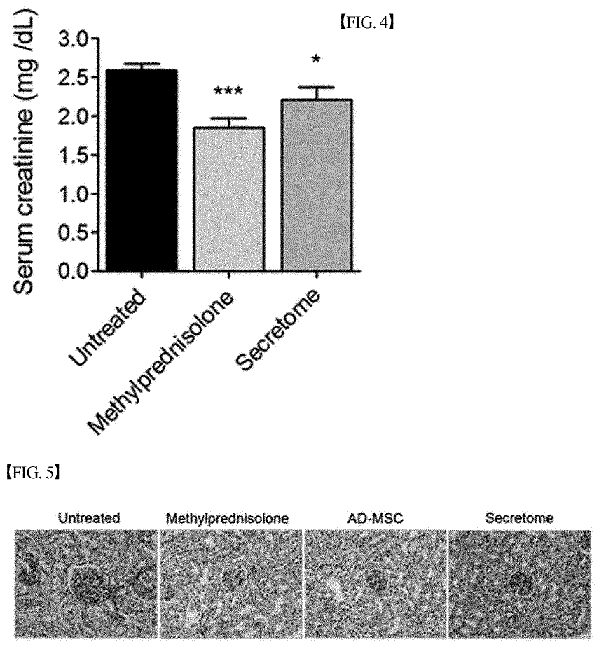

In addition, as shown in FIG. 4, proteinuria in the group treated with the secretome according to the present disclosure decreased to an extent similar to that in the group treated with methylprednisolone. Thus, it could be seen that the secretome had an anti-inflammatory effect.

The concentration of creatinine in the serum represents kidney function. As shown in FIG. 5, it could be confirmed that the content of creatinine in the group treated with the secretome according to the present disclosure significantly decreased compared to that in the untreated group.

### [Example 3] Effect of Protection Against Kidney Tissue Damage in Lupus-Induced Mouse Models

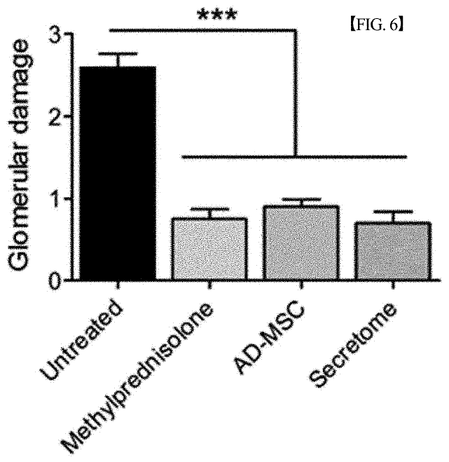

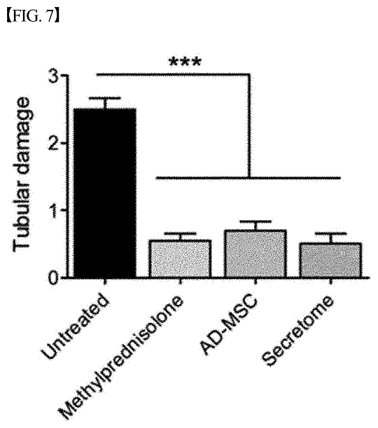

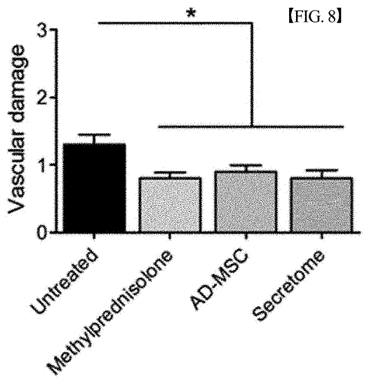

After performing the experiment in the same manner as in Example 2 above, the lupus nephritis mouse models were euthanized, and then the kidney tissues were fixed in formalin, embedded in paraffin, sectioned thinly, and then subjected to PAS staining. The results of the staining are shown in FIG. 5. In addition, the extents of glomerular damage, tubular damage and vascular damage in the kidney tissue of each treatment group were evaluated, and the results of the evaluation are shown in FIGS. 6 to 8, respectively.

The expression levels of IgG and C3, which are deposited in kidney tissue at the onset of lupus nephritis, were analyzed by fluorescence staining. The kidney tissues, treated with an OCT compound and stored at −20° C., were sectioned thinly, and then treated with anti-mouse IgG and anti-mouse C3 antibodies and additionally treated with secondary fluorescent antibodies. Next, the sections were imaged under confocal microscopy and the fluorescence intensities thereof were quantitatively analyzed. The results of the analysis are shown in FIG. 9.

As shown in FIGS. 5 to 8, it could be confirmed that when the mice were treated with the secretome according to the present disclosure, the kidney tissue was more protected from damage than when the mice were treated with methylprednisolone or mesenchymal stem cells.

As shown in FIG. 9, it could be confirmed that the expression levels of IgG and C3 in the kidney tissue of the group treated with the secretome according to the present disclosure significantly decreased compared to those in the untreated group and the expression levels decreased compared to those in the group treated with mesenchymal stem cells.

### [Example 4] Changes in Size of Spleen and Number of Splenocytes in Lupus-Induced Mouse Models

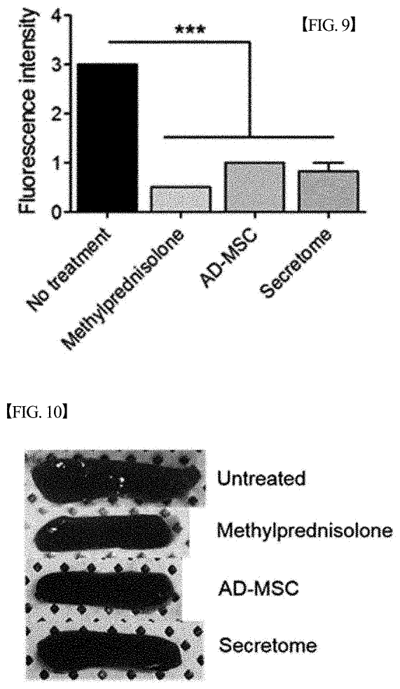

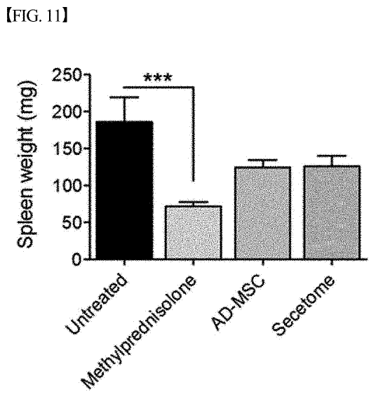

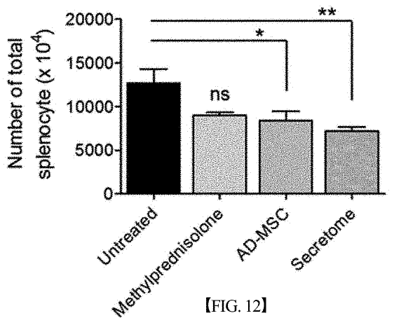

After performing the experiment in the same manner as Example 2 above, the lupus nephritis mouse models were euthanized, and then the spleen tissues were imaged. The results of the imaging are shown in FIG. 10. The weight of the spleen in each treatment group was measured, and the results of the measurement are shown in FIG. 11. The number of splenocytes in each treatment group was measured, and the results of the measurement are shown in FIG. 12.

As shown in FIGS. 10 and 11, it could be confirmed that the size of the enlarged spleen in the lupus nephritis mouse models significantly decreased when treated with the secretome according to the present disclosure. As shown in FIG. 12, it could be confirmed that the increased number of splenocytes in the lupus nephritis mouse models was also more decreased when treated with the secretome according to the present disclosure than when treated with methylprednisolone or mesenchymal stem cells.

### [Example 5] Change in Expression Level of CD4+ T Cells in Lupus-Induced Mouse Models

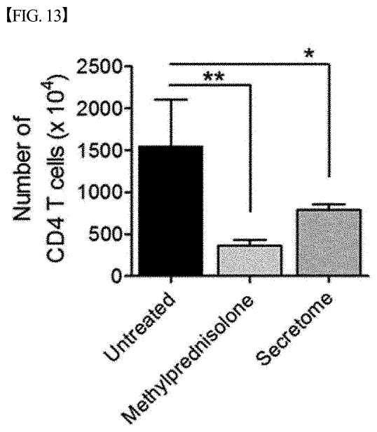

After performing the experiment in the same manner as Example 2 above, the expression level of CD4+ T cells in the splenocytes of the lupus nephritis mouse models was measured using a flow cytometer, and the results of the measurement are shown in FIG. 13.

As shown in FIG. 13, it could be confirmed that the increased expression level of CD4+ T cells in the lupus nephritis mouse models significantly decreased when treated with the secretome according to the present disclosure. Thus, it could be seen that the secretome had an anti-inflammatory effect.

### [Example 6] Analysis of T Cells in Lupus-Induced Mouse Models

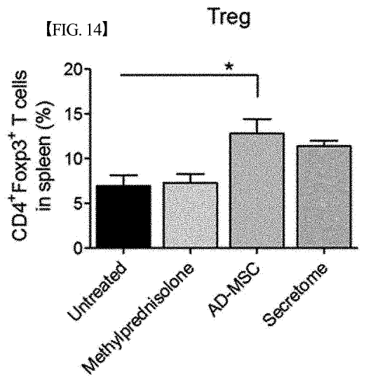

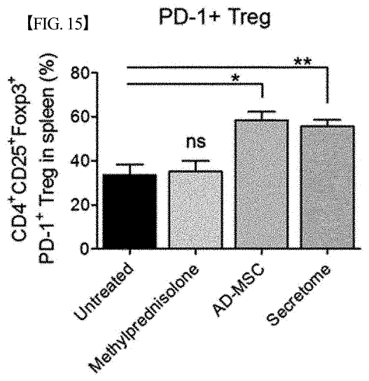

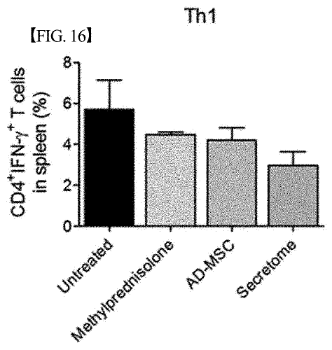

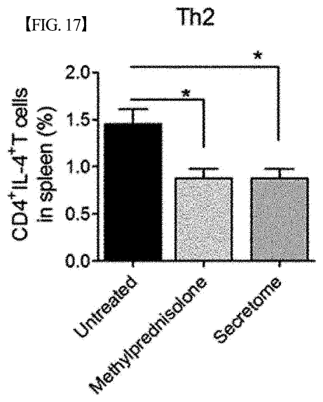

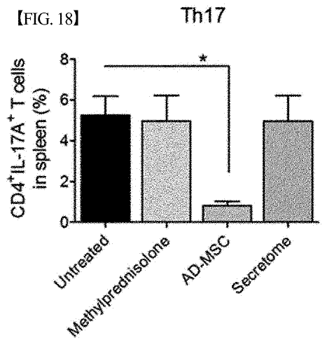

After performing the experiment in the same manner as Example 2 above, the expression levels of CD4+Foxp3+ cells (corresponding to regulatory T (Treg) cells), CD4+CD25+Foxp3+PD-1+ cells, CD4+IFN-γ+ cells (corresponding to Th1 cells), CD4+IL-4+ cells (corresponding to Th2 cells) and CD4+IL-17+ cells (corresponding to Th17 cells) in the splenocytes of the lupus nephritis mouse models were analyzed using a flow cytometer, and the results of the analysis are shown in FIGS. 14 to 18.

As shown in FIGS. 14 to 18, the expression levels of the Th1 and Th2 cells significantly decreased when the lupus nephritis mouse models were treated with the secretome according to the present disclosure compared to when untreated or treated with methylprednisolone, and the expression levels of the Treg cells (CD4+Foxp3+ cells and CD4+CD25+Foxp3+PD-1+ cells significantly increased when the lupus nephritis mouse models were treated with the secretome according to the present disclosure. That is, it could be confirmed that the secretome according to the present disclosure controls the function of inflammatory cells, Th1 cells and Th2 cells, but induces the function of Treg cells that control inflammatory cells.

Meanwhile, the expression level of Th17 cells in the group treated with the secretome according to the present disclosure was maintained at the same level as that in the untreated group, but decreased in the group treated with the mesenchymal stem cells. Thus, it could be seen that the secretome according to the present disclosure and the mesenchymal stem cells acted by different mechanisms.

### [Example 7] Analysis of B Cells in Lupus-Induced Mouse Models

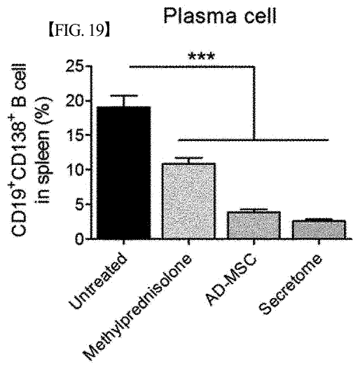

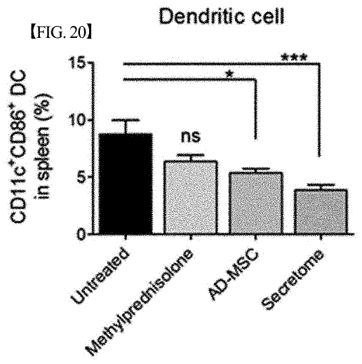

After performing the experiment in the same manner as Example 2 above, the expression level of CD19+CD138+ cells (corresponding to B cells and plasma B cells) in the splenocytes of the lupus nephritis mouse models was analyzed using a flow cytometer, and the results of the analysis are shown in FIG. 19.

As shown in FIG. 19, it could be confirmed that when the lupus nephritis mouse models were treated with the secretome according to the present disclosure, the expression level of B cells significantly decreased compared to when untreated or treated with methylprednisolone, and more greatly decreased than when treated with the mesenchymal stem cells.

### [Example 8] Analysis of Dendritic Cells and M1 Cells in Lupus-Induced Mouse Models

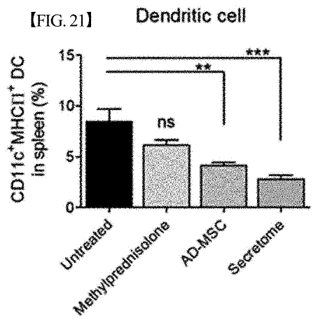

After performing the experiment in the same manner as Example 2 above, the expression levels of CD11c+CD86+ cells and CD11c+MHCII+ cells (corresponding to dendritic cells) in the splenocytes of the lupus nephritis mouse models were analyzed using a flow cytometer, and the results of the analysis are shown in FIGS. 20 and 21.

The CD86 and MHCII are markers indicating the activity of dendritic cells. As shown in FIGS. 20 and 21, it could be confirmed that when the lupus nephritis mouse models were treated with the secretome according to the present disclosure, the activity of dendritic cells significantly decreased when untreated or treated with methylprednisolone, and the activity more decreased than when the lupus nephritis mouse models were treated with the mesenchymal stem cells.

### [Example 9] Analysis of Macrophages in Lupus-Induced Mouse Models

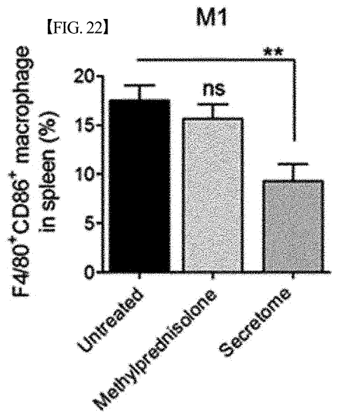

After performing the experiment in the same manner as Example 2 above, the expression level of F4/80+CD86+ cells (inflammatory macrophages) in the splenocytes of the lupus nephritis mouse models was analyzed using a flow cytometer, and the results of the analysis are shown in FIG. 22.

As shown in FIG. 22, it could be confirmed that when the lupus nephritis mouse models were treated with the secretome according to the present disclosure, the activity of the inflammatory macrophages significantly decreased compared to when untreated or treated with methylprednisolone.

### [Example 10] Analysis of Number of Lymphocytes in Lupus-Induced Mouse Models

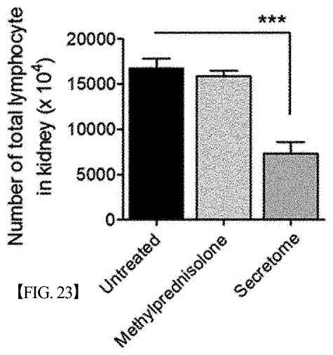

After performing the experiment in the same manner as Example 2 above, the number of total lymphocytes in the kidney tissues of the lupus nephritis mouse models was measured using a flow cytometer, and the results of the measurement are shown in FIG. 23.

As shown in FIG. 23, it could be confirmed that when the lupus nephritis mouse models were treated with methylprednisolone, the number of lymphocytes was almost similar to that in the untreated group, but when the lupus nephritis mouse models were treated with the secretome according to the present disclosure, the number of lymphocytes significantly decreased.

### [Example 11] Analysis of Expression Levels of Cytokines in Serum in Lupus-Induced Mouse Models

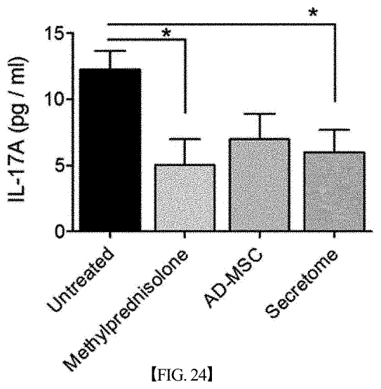

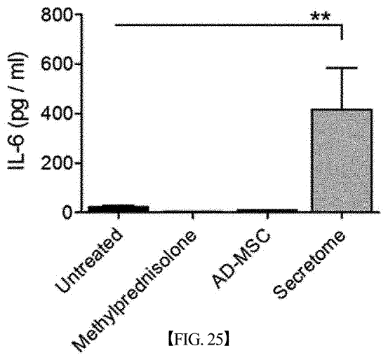

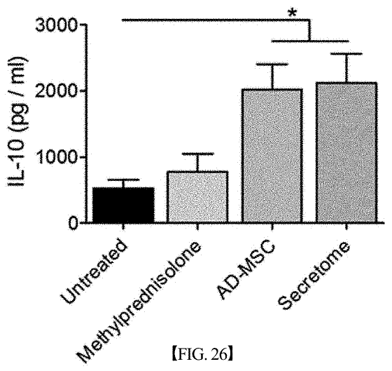

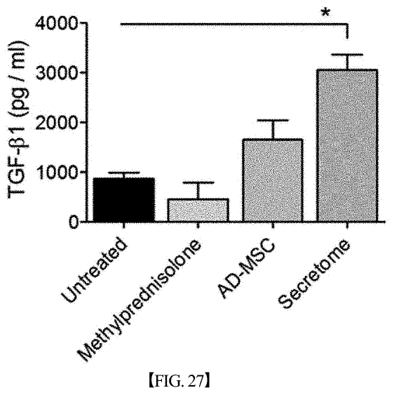

After performing the experiment in the same manner as Example 2 above, the expression levels of the cytokines IL-17, IL-6, IL-10 and TGF-β1 in the sera of the lupus nephritis mouse models were measured by an FT ISA assay, and the results of the measurement are shown in FIGS. 24 to 27, respectively.

As shown in FIGS. 24 to 27, it could be confirmed that when the lupus nephritis mouse models were treated with the secretome according to the present disclosure, the expression levels of IL-17 in the serum decreased compared to when untreated or treated with the mesenchymal stem cells, and the expression levels of IL-6, IL-10, and TGF-β1 increased.

### [Example 12] Analysis of Double-Stranded DNA in Serum in Lupus-Induced Mouse Models

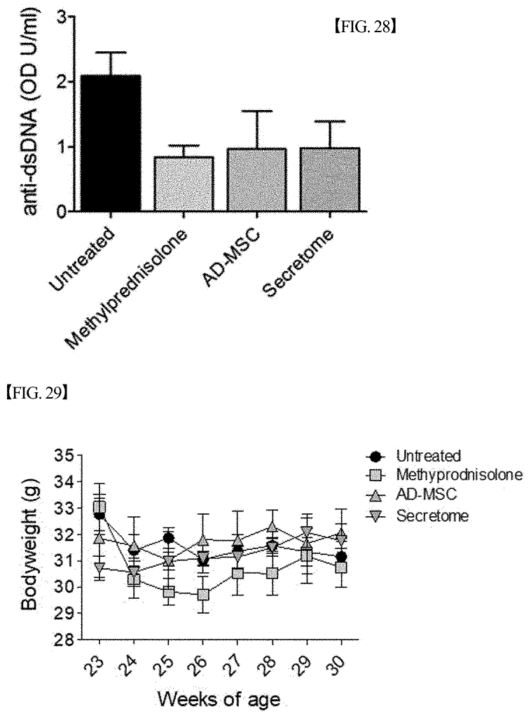

After performing the experiment in the same manner as Example 2 above, the expression level the autoantibody anti-dsDNA, which is expressed at the onset of lupus, in the sera of the lupus nephritis mouse models, was measured by an FT ISA assay, and the results of the measurement are shown in FIG. 28.

As shown in FIG. 28, it could be confirmed that when the lupus nephritis mouse models were treated with the secretome according to the present disclosure, the expression level of anti-dsDNA in the serum significantly decreased compared to when untreated.

### [Example 13] Analysis of Change in Body Weight in Lupus-Induced Mouse Models

According to the same method as Example 2 above, the lupus nephritis mouse models were treated with each of the secretome, isolated and concentrated according to one embodiment of the present disclosure, and adipose-derived mesenchymal stem cells (AD-MSCs) or methylprednisolone as a positive treatment control. Then, changes in the body weights of the mouse models were measured, and the results of the measurement are shown in FIG. 29.

As shown in FIG. 29, it could be confirmed that when the lupus nephritis mouse models were untreated or treated with methylprednisolone, the body weight decreased, but when the lupus nephritis mouse models were treated with the secretome according to the present disclosure, the body weight increased as the treatment period increased.

In general, when mesenchymal stem cells are administered, not only a sufficient supply thereof is difficult, but also when these cells are transplanted in vivo, the possibility of allogeneic transplantation rejection and tumorigenesis may become problematic. However, the secretome derived from mesenchymal stem cells according to the present disclosure may be produced and synthesized in large scale from a cell line, and has no immunogenicity. In addition, from the in vivo experiment as described above, it can be seen that the secretome has a therapeutic effect equivalent to or greater than mesenchymal stem cells against lupus, particularly lupus nephritis. Furthermore, from the results that the regulation of expression level of Th17 cells in the spleen and expression of IL-6 in the serum differs between the mesenchymal stem cell-derived secretome and the mesenchymal stem cells, it can be seen that the mechanisms of the therapeutic effects of the mesenchymal stem cell-derived secretome and the mesenchymal stem cells differ from each other.

### INDUSTRIAL APPLICABILITY

The present disclosure is directed to a medicament capable of effectively preventing, ameliorating or treating lupus using a secretome derived from mesenchymal stem cells.

1-18. (canceled)

19. A method for preventing or treating lupus, comprising a step of administering a target subject a secretome derived from mesenchymal stem cells in order to prevent or treat lupus.

20. The method of claim 19, wherein the secretome is isolated from a culture obtained by culturing the mesenchymal stem cells.

21. The method of claim 20, wherein the culturing of the mesenchymal stem cells is performed by culturing the mesenchymal stem cells in a mesenchymal stem cell culture medium for 24 to 96 hours, and then culturing the mesenchymal stem cells in a serum-free medium for 24 to 72 hours.

22. The method of claim 21, wherein the mesenchymal stem cell culture medium is any one selected from the group consisting of a Dulbecco's modified Eagle's medium (DMEM) containing 5 to 15 wt % of fetal bovine serum (FBS) and 0.05 to 0.2 mM of mercaptoethanol, RPMI-1640 medium, StemPro medium, MSCGro medium, MesenCult medium, and Nutri Stem medium.

23. The method of claim 20, wherein the secretome is a concentrate obtained after centrifuging the culture of the mesenchymal stem cells at 500 to 1,500 xg and recovering a supernatant of the centrifuged culture.

24. The method of claim 23, wherein the concentrate is obtained by a step of filtering the supernatant through a 0.1 to 0.3 μm filter, and a step of filtering molecules of 3 kDa or less in size.

25. The method of claim 24, wherein the filtering of the molecules of 3 kDa or less in size is performed by diafiltration using a tangential flow filtration (TFF) system.

26. The method of claim 23, wherein the concentrate is obtained by reacting the recovered supernatant with a polar alcohol solvent.

27. The method of claim 26, wherein the reaction of the supernatant with the polar alcohol solvent is performed at a temperature of −30 to 0° C. for 5 to 500 minutes.

28. The method of claim 26, wherein the polar alcohol solvent is mixed with the supernatant in an amount of 2 to 5 times the weight of the supernatant.

29. The method of claim 23, wherein the concentrate is a freeze-dried concentrate.

30. A method for preventing or ameliorating lupus, comprising a step of administering a target subject a secretome derived from mesenchymal stem cells in order to prevent or ameliorate lupus.

31. The method of claim 30, wherein the secretome is isolated from a culture obtained by culturing the mesenchymal stem cells.

32. The method of claim 31, wherein the culturing of the mesenchymal stem cells is performed by culturing the mesenchymal stem cells in a mesenchymal stem cell culture medium for 24 to 96 hours, and then culturing the mesenchymal stem cells in a serum-free medium for 24 to 72 hours.

33. The method of claim 32, wherein the mesenchymal stem cell culture medium is any one selected from the group consisting of a Dulbecco's modified Eagle's medium (DMEM) containing 5 to 15 wt % of fetal bovine serum (FBS) and 0.05 to 0.2 mM of mercaptoethanol, RPMI-1640 medium, StemPro medium, MSCGro medium, MesenCult medium, and NutriStem medium.

34. The method of claim 31, wherein the secretome is a concentrate obtained after centrifuging the culture of the mesenchymal stem cells at 500 to 1,500 xg and recovering a supernatant of the centrifuged culture.

35. The method of claim 34, wherein the concentrate is obtained by a step of filtering the supernatant through a 0.1 to 0.3 μm filter, and a step of filtering molecules of 3 kDa or less in size.

36. The method of claim 35, wherein the filtering of the molecules of 3 kDa or less in size is performed by diafiltration using a tangential flow filtration (TFF) system.
